(19) United States
(12) Patent Application Publication
(10) Pub. No.: US 2023/0149490 A1
(43) Pub. Date: May 18, 2023

(54) NOVEL COMPOSITION COMPRISING EDELWEISS-DERIVED EXOSOME AS ACTIVE INGREDIENT

(71) Applicant: ExoCoBio Inc., Seoul (KR)

(72) Inventor: Byong Seung CHO, Gunpo-si (KR)

(73) Assignee: ExoCoBio Inc., Seoul (KR)

(21) Appl. No.: 18/155,170

(22) Filed: Jan. 17, 2023

Related U.S. Application Data
(63) Continuation of application No. PCT/KR21/06949, filed on Jun. 3, 2021.

(30) Foreign Application Priority Data
Sep. 29, 2020 (KR) 10-2020-0126994
Mar. 16, 2021 (KR) 10-2021-0034327
Apr. 7, 2021 (KR) 10-2021-0045494

(51) Int. Cl. A61K 36/28; A61K 8/9789; A61P 17/02; A61P 29/00; A61Q 19/08

(52) U.S. Cl. CPC A61K 36/28; A61K 8/9789; A61P 17/02; A61P 29/00; A61Q 19/08; A61K 2236/11; A61K 2236/53

(57) ABSTRACT

A composition containing edelweiss-derived exosomes as an active ingredient is provided for skin elasticity improvement, skin wrinkle reduction, skin regeneration, anti-inflammation, wound healing or wound healing acceleration. The composition is less likely to contain impurities such as growth regulation factors than conditioned media of edelweiss callus, or filtrates or extracts thereof, and has excellent effects on skin elasticity improvement, skin wrinkle reduction, skin regeneration, anti-inflammation, wound healing and/or wound healing acceleration.

### CROSS REFERENCE

This application is a Bypass Continuation of International Application No. PCT/KR2021/006949 filed Jun. 3, 2021, claiming priority based on Korean Patent Application No. 10-2020-0126994 filed Sep. 29, 2020, Korean Patent Application No. 10-2021-0034327 filed Mar. 16, 2021 and Korean Patent Application No. 10-2021-0045494 filed Apr. 7, 2021, the entire contents of which are incorporated herein by reference.

### TECHNICAL FIELD

The present invention relates to a novel composition comprising edelweiss-derived exosomes as an active ingredient, in particular, a composition for skin elasticity improvement, skin wrinkle reduction, skin regeneration, anti-inflammation, wound healing or wound healing acceleration comprising edelweiss-derived exosomes as an active ingredient.

Moreover, the present invention relates to a cosmetic composition for skin elasticity improvement, skin wrinkle reduction or skin regeneration and a pharmaceutical composition for anti-inflammation, wound healing or wound healing acceleration, containing the aforesaid composition.

### BACKGROUND ART

It is known that skin aging leads to a decrease in skin elasticity and an increase in skin wrinkles and that the decrease in skin elasticity and the formation of skin wrinkles occur due to decreased synthesis of collagen and stimulated expression of the collagenase matrix metalloproteinase (MMP).

In addition, it is known that in skin cells, COX-2, an enzyme that produces inflammatory cytokines increases due to aging progression or ultraviolet (UV) rays, resulting in increased synthesis of prostaglandin E2 and increased production of inflammation inducers. Due to inflammatory reactions, the biosynthesis of MMP increases, causing collagen degradation and resulting in the decrease in skin elasticity and the formation of skin wrinkles. In particular, when sunlight and ultraviolet rays are irradiated directly onto skin, a lot of free radicals are generated, and these free radicals could damage the antioxidant defense system of skin, thus increasing wrinkles, making skin loose and accelerating skin aging. Substances known to be effective in reducing skin wrinkles include adenosine and retinoic acid. However, adenosine has little efficacy in clinical practice, and retinoic acid cannot be used for pregnant women and has side effects such as erythema.

Inflammation is a defense response of the body against physical or chemical injury, infection of bacteria, fungi or viruses, or pathological conditions caused by various allergens and the like. Inflammatory response appears as part of innate immune response. Various substances and physiological and chemical phenomena are involved in inflammatory response, and recent studies have shown that various inflammatory cytokines play an important role in inflammatory response. Major cytokines involved in inflammatory response include IL-1β, TNF-α, IL-6, IL-8, IL-12, IFN-β, and the like. The increased expression and secretion of these cytokines and the activation thereof are associated with a series of complex physiological responses, including secretion of inflammatory mediators, immune cell infiltration, cell migration, and tissue destruction, as well as symptoms such as erythema, edema, fever and pain.

In general, inflammatory response does not become a significant problem and the affected area returns to its normal state, if the infectious agent is removed from the body and the damaged tissue is regenerated. However, if the infectious agent is not removed from the body or the inflammatory response is excessive or persistent due to internal substances of the body, acute or chronic inflammatory disease occurs. Non-steroidal anti-inflammatory drugs, steroidal anti-inflammatory drugs, antagonists of neuropeptides, COX inhibitors, anti-histamines, and immunosuppressive drugs such as cyclosporine A are used for alleviation or treatment of inflammatory response or inflammatory diseases caused thereby, but have problems that they cause adverse effects such as skin atrophy, vasodilation, depigmentation, hypersensitivity reactions, tolerance, neutropenia and the like. In addition, there is a limit that the aforesaid drugs merely help to control symptoms related to inflammation to a certain level rather than the underlying treatment therefor.

Recently, there have been reports that cell secretomes contain various bioactive molecules that regulate cellular behaviors. In particular, cell secretomes contain ‘exosome’ that has intercellular signaling functions, and thus studies on the components and functions thereof have been actively conducted.

Cells release various membraneous vesicles to their extracellular environment, and these released vesicles are usually called extracellular vesicles (EVs). The EV is also called cell membrane-derived vesicle, ectosome, shedding vesicle, microparticle, exosome, etc., and is also used discriminately from exosome in some cases.

Exosome is a vesicle of tens to hundreds of nanometers in size, which comprises a phospholipid bilayer membrane having the same structure as that of the cell membrane. This exosome contains proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosome is an intercellular signaling mediator secreted by cells, and various cellular signals transmitted through it regulate cellular behaviors, including the activation, growth, migration, differentiation, dedifferentiation, apoptosis, and necrosis of target cells. Exosome contains specific genetic materials and bioactive factors depending on the nature and state of cells from which the exosome was derived. Exosome derived from proliferating stem cells regulates cell behaviors such as cell migration, proliferation and differentiation, and recapitulates the function of stem cells involved in tissue regeneration (Nature Review Immunology 2002 (2) 569-579).

That is, exosomes called “avatars” of cells contain bioactive factors such as growth factors, similar to cells, and serve as carriers that transmit bioactive factors between cells, that is, serve to mediate cell-to-cell communication. Exosomes are known to be released not only from animal cells such as stem cells, immune cells, fibroblasts and cancer cells, but also from cells of various organisms such as plants, bacteria, fungi, and algae. For example, exosomes may be isolated from conditioned media of plant stem cells, as well as conditioned media of cancer cells, immune cells, mesenchymal stem cells, and the like.

However, studies on the isolation, purification and characterization of exosomes derived from plant stem cells are still in their early stages. Most of them are at a level where they simply called extracellular vesicles mixed in the filtrate of plant juice as exosomes and are merely used for marketing purposes. Therefore, more detailed characterization and functional studies of exosomes derived from plant stem cells are required.

Edelweiss is one of the most famous alpine plants in the Alps. It grows well in dry places and is distributed in Europe, Siberia, the Himalayas, China, Korea, Japan and Sakhalin. Since edelweiss has many flavonoids such as vitamin E, phenolic acid, luteolin and luteolin-7-glucoside, it has skin soothing, antioxidant and UV protection effects. However, technologies to date are at a level where filtrates obtained by drying edelweiss leaves, stems or roots, immersing the dried leaves, stems or roots in water, infusing them and filtering the infusion, or extracts obtained by hot water-extracting or solvent-extracting edelweiss leaves, stems or roots are used for raw materials of food or cosmetic products.

In addition, a technology of using conditioned media of edelweiss callus obtained by culturing edelweiss callus induced by wounding leaves of edelweiss has been introduced, but this technology is also merely at a level where conditioned media of callus themselves or extracts obtained by drying the callus and then subjecting the dried callus to hot-water extraction or solvent extraction are used as cosmetic ingredients. Further, conditioned media of edelweiss callus contain growth regulators or a callus inducing substance, and thus are hardly regarded as natural cosmetic ingredients, and the growth regulators contained in the cultures may cause side effects such as skin troubles.

The present inventor has found that exosomes derived from edelweiss are effective in skin elasticity improvement, skin wrinkle reduction, skin regeneration, anti-inflammation, wound healing or wound healing acceleration, and has developed a cosmetic composition for skin elasticity improvement, skin wrinkle reduction or skin regeneration containing edelweiss-derived exosomes as an active ingredient, and a pharmaceutical composition for anti-inflammation, wound healing or wound healing acceleration containing edelweiss-derived exosomes as an active ingredient.

Meanwhile, it is to be understood that the matters described as the background art are intended merely to aid in the understanding of the background of the present invention and are not admitted as prior art against the present invention.

### SUMMARY OF INVENTION

An object of the present invention is to provide a composition for skin elasticity improvement, skin wrinkle reduction, skin regeneration, anti-inflammation, wound healing or wound healing acceleration comprising edelweiss-derived exosomes as an active ingredient.

Another object of the present invention is to provide a cosmetic composition for skin elasticity improvement, skin wrinkle reduction or skin regeneration and a pharmaceutical composition for anti-inflammation, wound healing or wound healing acceleration, containing the aforesaid composition.

However, the objects of the present invention as described above are illustrative and the scope of the present invention is not limited thereby. In addition, other objects and advantages of the present invention will be more apparent from the following description, the appended claims and the accompanying drawings.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows particle size distribution and number of particles obtained by transmitted electron microscopy (TEM) of edelweiss-derived exosomes of the present invention.

FIG. 2 depicts fluorescence microscopic images of cells showing that fluorescence-stained exosomes are delivered into human dermal fibroblasts (green: exosomes delivered into cells; and blue: cell nucleus).

FIG. 3 is a graph showing the relative amount of collagen increases after human dermal fibroblasts are treated with edelweiss-derived exosomes.

FIGS. 4A to 4C are graphs showing that the migration of human dermal fibroblasts remarkably increased after scratch-wounds of human dermal fibroblasts were treated with edelweiss-derived exosomes.

FIG. 5 is a graph showing that IL-6 induced in RAW 264.7 cells by LPS remarkably decreased when RAW 264.7 cells were treated with edelweiss-derived exosomes.

FIG. 6 is a graph showing that IL-6 induced in RAW 264.7 cells by LPS remarkably decreased in a concentration-dependent manner when RAW 264.7 cells were treated with each concentration of edelweiss-derived exosomes.

### DETAILED DESCRIPTION OF INVENTION

The present invention provides a composition for skin elasticity improvement, skin wrinkle reduction, skin regeneration, anti-inflammation, wound healing or wound healing acceleration comprising edelweiss-derived exosomes as an active ingredient.

As used herein, the term “edelweiss” refers to plants belonging to the genus Leontopodium. For example, a plant belonging to the genus Leontopodium may be Leontopodium alpinum, Leontopodium andersonii, Leontopodium artemisiifolium, Leontopodium aurantiacum, Leontopodium brachyactis, Leontopodium calocephalum, Leontopodium campestre, Leontopodium chuii, Leontopodium conglobatum, Leontopodium coreanum, Leontopodium dedekensii, Leontopodium delavayanum, Leontopodium discolor, Leontopodium fangingense, Leontopodium fauriei, Leontopodium forrestianum, Leontopodium franchetii, Leontopodium giraldii, Leontopodium haastioides, Leontopodium hallaisanense, Leontopodium haplophylloides, Leontopodium hayachinense, Leontopodium himalayanum, Leontopodium jacotianum or Leontopodium japonicum, preferably, Leontopodium alpinum.

As used herein, the term “topical administration” for example means to apply or spread a composition that is suitable for topical application, onto the surface of skin including the scalp, not limited thereto. Thus, the “topical administration” includes administering a composition to the skin and/or scalp by topical routes such as injection, microneeding, spread, spray, transdermal delivery using a patch or a sheet, iontophoresis, and the like.

As used herein, the term “exosomes” refers to nano-sized vesicles secreted or released from plant cells into extracellular spaces and having a membrane structure, and is also referred to as exosome-like vesicles or exosome-like particles.

As used herein, the term “skin elasticity” refers to a feature in which skin deformed by an external force easily returns to its original shape when the external force is removed. The term “skin wrinkles” refers to fine lines caused by skin aging. Skin wrinkles may be caused by genetic factors, reduction in collagen and elastin present in the skin dermis, external environmental factors, or the like. Accordingly, the term “skin wrinkle reduction or improvement” as used herein refers to suppressing or inhibiting the formation of wrinkles on the skin, or reducing already formed wrinkles.

As used herein, the term “anti-inflammation” means prevention, suppression, alleviation, amelioration or treatment of inflammation. As an example, not limiting the present invention, examples of the inflammatory diseases include dermatitis, atopic dermatitis, eczema, inflammation caused by bacterial, viral or fungal infections, burns, inflammation caused by burns, wounds, inflammation caused by wounds, and the like.

As used herein, the term “wound” means a condition in which a part or all of the body is injured, and is intended to encompass pathological conditions in which a tissue constituting an inside or an external surface of the body, for example, skin, muscle, nerve tissue, bone, soft tissue, internal organ, or blood vessel tissue, is damaged or destroyed. As an example, not limiting the present invention, examples of the wound include abrasion, laceration, stab wound, incised wound, avulsion, bedsore, tissue destruction caused by irradiation, penetrated wound, gunshot wound, burn, frostbite, surgical wound, sutures after plastic surgery, wound caused by chemical substance and so on, and may include any damage to any part of an individual.

As used herein, the term “iontophoresis” refers to a method of flowing a microcurrent through a skin to which an active ingredient has been applied, generating a potential difference thereby and changing the electrical environment of the skin, and thus allowing an ionized active ingredient to penetrate the skin by electrical repulsion. Examples of iontophoresis that is used in one embodiment of the present invention include: a method of introducing a microcurrent into a skin by allowing the microcurrent to flow from an external power source into an electrode patch on the skin, the microcurrent generated by the external power source; a method of introducing a microcurrent into a skin, the microcurrent generated by a battery provided in an electrode patch on the skin; and a method of introducing a microcurrent into a skin through a patch on the skin provided with a reverse electrodialysis device, the microcurrent generated by the concentration difference between high concentration electrolyte solution and low concentration electrolyte solution in the reverse electrodialysis device. However, the present invention is not limited thereto, and various types of iontophoresis may, of course, be used.

As used herein, the term “edelweiss-derived exosomes” is meant to include all exosomes isolated from, for example, a conditioned medium of edelweiss plant cells, a conditioned medium of edelweiss callus, or a biological solution of edelweiss equivalent thereto, or derived from plant cells or plant stem cells of edelweiss, for example, secreted and/or released from the plant cells or the plant stem cells.

The composition containing edelweiss-derived exosomes as an active ingredient according to one embodiment of the present invention may exhibit at least one effect selected from the group consisting of skin elasticity improvement, skin wrinkle reduction, skin regeneration, anti-inflammation, wound healing or wound healing acceleration.

In the composition according to one embodiment of the present invention, the edelweiss-derived exosomes may be isolated and purified from a conditioned medium of plant cells obtained from a leaf or a stem tissue of edelweiss, or a conditioned medium of edelweiss callus induced by making a wound on a cotyledon germinated from an edelweiss seed.

The composition containing edelweiss-derived exosomes as an active ingredient according to one embodiment of the present invention may be a cosmetic composition or a pharmaceutical composition.

The present invention provides a cosmetic composition for skin elasticity improvement, skin wrinkle reduction or skin regeneration comprising edelweiss-derived exosomes as an active ingredient. The cosmetic composition may be, for example, a shampoo, a soap, a rinse, a surfactant-containing cleanser, a cream, a lotion, an ointment, a tonic, a treatment, a conditioner, a suspension, an emulsion, a paste, a gel, an oil, a wax, a spray, an aerosol, a mist or a power, preferably, a cream or a lotion.

In addition, the present invention provides a pharmaceutical composition for anti-inflammation, wound healing or wound healing acceleration comprising edelweiss-derived exosomes as an active ingredient.

As an example, not limiting the present invention, the pharmaceutical composition according to one embodiment of the present invention may be administered or treated by injection, microneedling, iontophoresis, application, or a combination thereof. For example, the pharmaceutical composition may be an injectable formulation, an infusion formulation, a spray formulation, a liquid formulation, or a patch formulation.

In one embodiment of the present invention, when the composition according to one embodiment of the present invention is used as a pharmaceutical composition, it may include pharmaceutically acceptable carriers, excipients or diluents. The carriers, excipients and dilutes include, but are not limited to, lactose, dextrose, trehalose, sucrose, sorbitol, mannitol, xylitol, erythritol, maltitol, starch, acacia gum, alginate, gelatin, calcium phosphate, calcium carbonate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. In addition, the effective amount of the pharmaceutical composition according to one embodiment of the present invention means the amount required for administration in order to achieve the effects of anti-inflammation, wound healing or wound healing acceleration. The content of the pharmaceutical composition according to one embodiment in a formulation may be suitably selected depending on the kind, amount, form and the like of additional components as described above. For example, the pharmaceutical composition of the present invention may be contained in an amount of about 0.1 to 99 wt %, preferably about 10 to 90 wt %, based on the total weight of an injectable formulation. Furthermore, the suitable dose of the pharmaceutical composition according to one embodiment of the present invention may be adjusted depending on the severity of disease, the type of formulation, formulating method, patient's age, sex, body weight, health condition, diet, excretion rate, the period of administration, and the regime of administration. For example, when the pharmaceutical composition according to one embodiment of the present invention is administered to an adult, it may be administered once to several times at a dose of 0.001 mg/kg to 100 mg/kg per day.

Meanwhile, when the composition according to one embodiment of the present invention is prepared as a cosmetic composition, it may suitably contain components which are generally used in cosmetic products, for example, moisturizers, antioxidants, oily components, UV absorbers, emulsifiers, surfactants, thickeners, alcohols, powder components, colorants, aqueous components, water, and various skin nutrients, etc., as needed, within the range that does not impair the effect of the present invention.

In addition, the cosmetic composition according to one embodiment of the present invention may include, in addition to the edelweiss-derived exosomes, an agent for improving skin condition and/or a moisturizer, which have been used in the prior art, within the range that does not impair the effects (e.g., skin condition improvement, skin elasticity improvement, skin wrinkle improvement or skin regeneration, etc.).

The cosmetic composition according to one embodiment of the present invention may be used in various forms, for example, a patch, a mask pack, a mask sheet, a cream, a tonic, an ointment, a suspension, an emulsion, a paste, a lotion, a gel, an oil, a pack, a spray, an aerosol, a mist, a foundation, a powder, and an oilpaper.

The cosmetic composition is used for the purpose of skin condition improvement, skin elasticity improvement, skin wrinkle reduction, skin regeneration or the like, and the cosmetic composition may be prepared as any formulation which is generally prepared in the art. For example, it may be formulated as a patch, a mask pack, a mask sheet, a skin softener, a nutrition, an astringent lotion, a nourishing cream, a massage cream, an eye cream, a cleansing cream, an essence, an eye essence, a cleansing lotion, a cleansing foam, a cleansing water, a sunscreen, a lipstick, a soap, a shampoo, a surfactant-containing cleanser, a bath preparation, a body lotion, a body cream, a body oil, a body essence, a body cleanser, a hairdye, a hair tonic, etc., but is not limited thereto.

The cosmetic composition according to one embodiment of the present invention contains components which are commonly used in cosmetic composition. For example, the cosmetic composition may contain conventional adjuvants and carriers, such as antioxidants, stabilizers, solubilizers, vitamins, pigments, and fragrances. In addition, other components in each formulation for the cosmetic composition may be suitably selected without difficulty by those skilled in the art depending on the type or intended use of the cosmetic composition.

Another embodiment of the present invention provides a cosmetic method for regulating mammalian skin conditions, except for treatment purposes, using the cosmetic composition. In the cosmetic method of the present invention, the expression “regulating skin conditions” means improving skin conditions and/or prophylactically regulating skin conditions, and the expression “improving skin conditions” means a visually and/or tactilely perceivable positive change in the appearance and feeling of skin tissue. For example, the expression “improving skin conditions” may include skin wrinkle reduction, skin elasticity improvement or skin regeneration.

The cosmetic method according to one embodiment of the present invention includes: (a) applying the cosmetic composition directly to a mammalian skin; or (b) contacting or attaching a patch, a mask pack or a mask sheet, which has the cosmetic composition applied thereto or soaked therein, to the mammalian skin; or sequentially performing (a) and (b). In step (a), the cosmetic composition may be a lotion or a cream.

Alternatively, the cosmetic method according to one embodiment of the present invention may further comprise step (c) removing the patch, the mask pack or the mask sheet from the mammalian skin after step (b), and applying the cosmetic composition to the mammalian skin. In step (c), the cosmetic composition may be a lotion or a cream.

In the cosmetic method according to one embodiment of the present invention, the mammal may be humans, dogs, cats, rodents, horses, cattle, monkeys, or pigs.

In addition, the present invention provides a method for treating inflammation, healing wound or accelerating wound healing, the method comprising administering a therapeutically effective amount of the pharmaceutical composition to a mammal, or applying the pharmaceutical composition to a skin, an inflammatory area, or a wounded area. The mammal may be at least one mammal selected from the group consisting of humans, dogs, cats, rodents, horses, cattle, monkeys and pigs.

### ADVANTAGEOUS EFFECTS

The composition of the present invention is less likely to contain impurities such as growth regulation factors than conventional conditioned media of edelweiss callus, or filtrates or extracts thereof, and has excellent effects on skin elasticity improvement, skin wrinkle reduction, skin regeneration, anti-inflammation, wound healing and/or wound healing acceleration.

It should be understood that the scope of the present invention is not limited to the aforementioned effects.

### EXAMPLES

Hereinafter, the present invention will be described in more detail with reference to the following examples. However, the following examples are only to illustrate the present invention and are not intended to limit or restrict the scope of the present invention. Those that can be easily inferred by those skilled in the art from the detailed description and examples of the present invention are interpreted as falling within the scope of the present invention. References referred to in the present invention are incorporated herein by reference.

Throughout the present specification, it is to be understood that, when any part is referred to as “comprising” any component, it does not exclude other components, but may further include other components, unless otherwise specified.

### Example 1: Preparation of Conditioned Media of Edelweiss Callus

According to induction and culture methods of plant callus known in the art, an edelweiss callus was induced from edelweiss leaves, and cells of the induced callus were cultured. In addition, a callus having a good growth state was selected and cultured in a large amount, thereby preparing conditioned media of edelweiss callus.

For example, commercially available edelweiss seeds were immersed in ethanol, washed with sterile water and sterilized with an antiseptic solution, and the edelweiss seeds were germinated in an aseptic condition in order to induce an edelweiss callus. Edelweiss callus was induced by making a wound on the germinated cotyledon with a knife, and the induced callus was cultured in a culture dish. Then, the callus in a good growth state was selected and cultured in a flask in a large amount.

### Example 2: Preparation of Edelweiss-Derived Exosomes

The conditioned media of edelweiss callus prepared as described in Example 1 were purchased from Biospectrum Co., Ltd. (located in Gyeonggi-do, Korea and supplying conditioned media of Leontopodium Alpinum callus). The conditioned media of edelweiss callus were filtered through a 0.22 μm filter to remove impurities such as cell debris, waste products and large particles. Edelweiss-derived exosomes were isolated from the filtered conditioned media by tangential flow filtration (TFF) method.

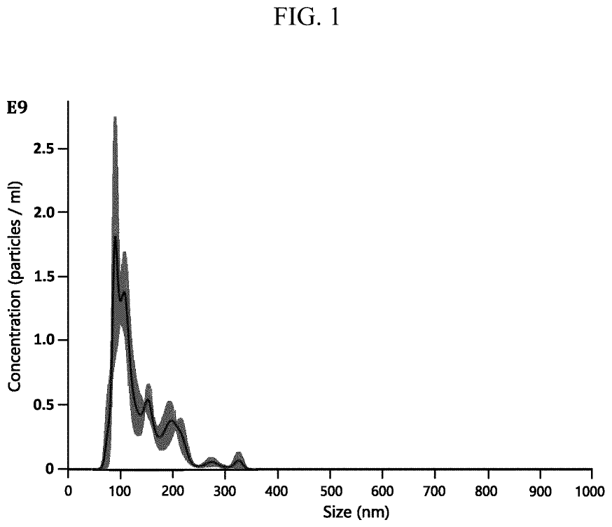

The size and concentration of the isolated edelweiss-derived exosomes were analyzed by nanoparticle tracking analysis (NTA) using NS300 (purchased from Malvern Panalytical) (FIG. 1).

### Example 3: Evaluation of Delivery Ability of Edelweiss-Derived Exosomes into Dermal Fibroblasts

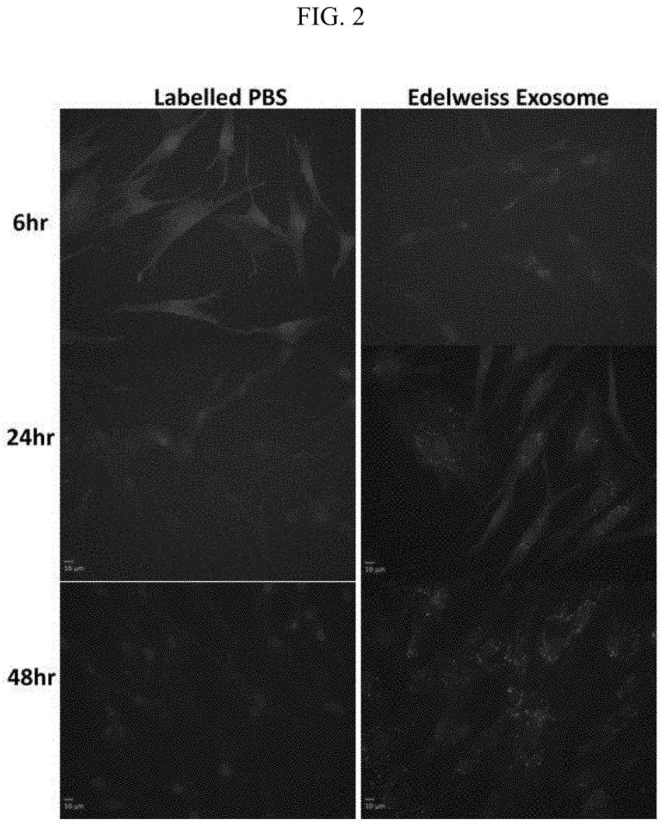

In order to examine whether the edelweiss-derived exosomes would be delivered into human dermal fibroblasts (purchased from CEFO Co., Ltd.), the following analysis was performed. To fluorescence-stain the membrane of the edelweiss-derived exosomes prepared in Example 2, the exosomes were allowed to react with PKH67 fluorescence dye (purchased from Sigma). After the reaction, the reaction solution was fractionated with an MW3000 column (purchased from ThermoFisher Scientific) to remove free PHK67 that was not stained in the exosome membrane. A negative control was prepared by allowing PKH67 fluorescence dye to react with a buffered solution and fractionating the reaction product with the MW3000 column. The exosomes stained with PKH67 were incubated with pre-cultured human dermal fibroblasts, and then whether the exosomes would be delivered into the cells over time was observed using a fluorescence microscope. Hoechst fluorescence dye (purchased from Sigma) was used to stain the cell nucleus. As a result of examining whether the exosomes would be delivered into the cells, it was confirmed that the fluorescence-stained exosomes were delivered into the cells and green fluorescence accumulated in the cells over time (FIG. 2).

### Example 4: Evaluation of Effect of Stimulating Collagen Production

Human dermal fibroblasts (purchased from CEFO Co., Ltd.) dispersed in DMEM medium containing fetal bovine serum were dispensed into a multiwell plate, and then cultured for 24 hours. Thereafter, the edelweiss-derived exosomes or the conditioned media of edelweiss callus prepared in Example 2 were diluted in serum-free medium, and then the human dermal fibroblasts were treated with each of the dilutions and cultured. To evaluate the collagen production effect using human dermal fibroblasts, experimental groups was classified as follows:

(1) Negative control (NC): an experimental group in which human dermal fibroblasts were treated with a serum-free medium alone;

(2) Conditioned media of edelweiss callus (CM): an experimental group in which human dermal fibroblasts were treated with the conditioned media of edelweiss callus, which were prepared in Example 2 and diluted with a serum-free medium (treatment concentration of CM based on the number of particles: 5.0×10⁸ particles/mL); and

(3) Edelweiss-derived exosomes (EXO): an experimental group in which human dermal fibroblasts were treated with the edelweiss-derived exosomes, which were prepared in Example 2 and diluted with a serum-free medium (treatment concentration of EXO based on the number of particles: 5.0×10⁸ particles/mL).

Thereafter, the treated human dermal fibroblasts were additionally cultured for 48 hours. The culture media were collected and centrifuged, and then the centrifuged media were prepared. The amount of collagen, which was synthesized from the human dermal fibroblasts and accumulated in the culture media, was measured using an EIA kit (purchased from Takara) for Procollagen Type I C-peptide (PIP). The measured amount of collagen was normalized by dividing it by the total number of cells measured using the MTT assay kit (purchased from Sigma) to determine the relative amount of collagen.

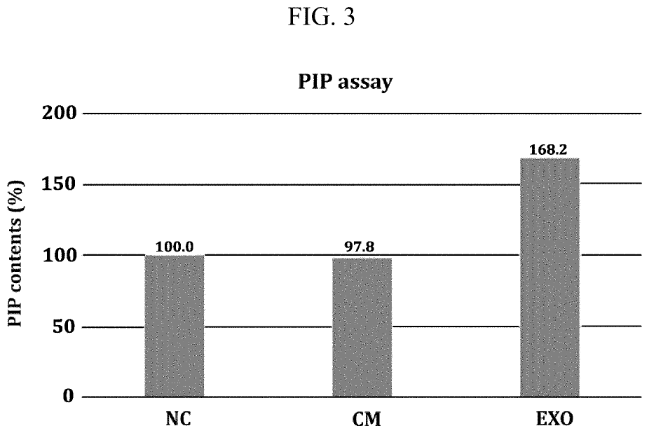

As shown in FIG. 3, the edelweiss-derived exosomes of the present invention increased collagen synthesis in human dermal fibroblasts compared to the negative control group, whereas the conditioned media of edelweiss callus did not increase collagen synthesis (FIG. 3).

As can be seen from the above results, the edelweiss-derived exosomes of the present invention have a useful functional activity (i.e., an activity of increasing collagen synthesis) in a cosmetic product for skin elasticity improvement, skin wrinkle reduction and/or skin regeneration. Thus, the edelweiss-derived exosomes of the present invention are useful as an active ingredient of a cosmetic composition for skin elasticity improvement, skin wrinkle reduction and/or skin regeneration.

### Example 5: Evaluation of Skin Regeneration Effect Using Dermal Fibroblasts

To evaluate whether the edelweiss-derived exosomes prepared as described in Example 2 promotes wound healing in human dermal fibroblasts, scratch-wound assay was performed. Human dermal fibroblasts dispersed in a DMEM containing fetal bovine serum were seeded into a culture plate for wound induction (ImageLock Plate; purchased from EssenBio) at a density of 5,000 cells/well and cultured for 24 hours under 5% CO₂ and 37° C. After the cells reached a confluency of 90% or more, scratches were made using a WoundMaker (purchased from EssenBio). To evaluate the skin regeneration effect using human dermal fibroblasts, experimental groups was classified as follows:

(1) Negative control (NC): an experimental group treated with a serum-free medium alone;

(2) Positive control (PC): an experimental group treated with a medium containing 10% fetal bovine serum;

(3) Conditioned media of edelweiss callus (CM): an experimental group treated with the conditioned media of edelweiss callus, which were prepared in Example 2 and diluted with a serum-free medium (treatment concentration of CM based on the number of particles: 1.0×10⁹ particles/mL); and

(4) Edelweiss-derived exosomes (EXO): an experimental group treated with the edelweiss-derived exosomes, which were prepared in Example 2 and diluted with a serum-free medium (treatment concentration of EXO based on the number of particles: 1.0×10⁹ particles/mL).

After that, each of the experimental groups was subjected to Scratch-Wound assay, and the human dermal fibroblasts were cultured at 37° C. under 5% CO₂ for 24 hours. At each of 6, 12 and 24 hours after the treatment of each experimental group, the wound healing efficacy in each experimental group was measured using Incucyte (purchased from Sartorius), respectively.

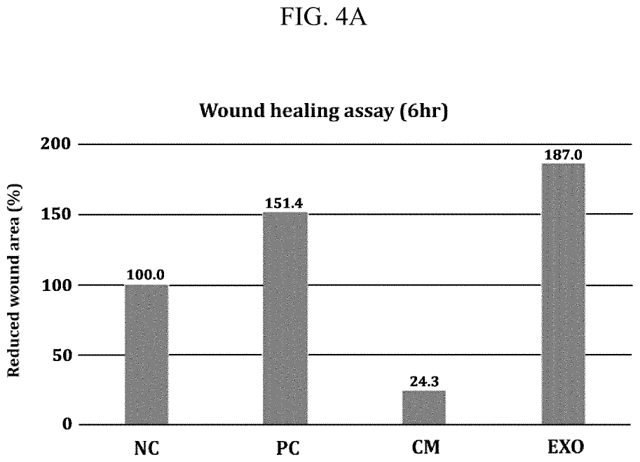

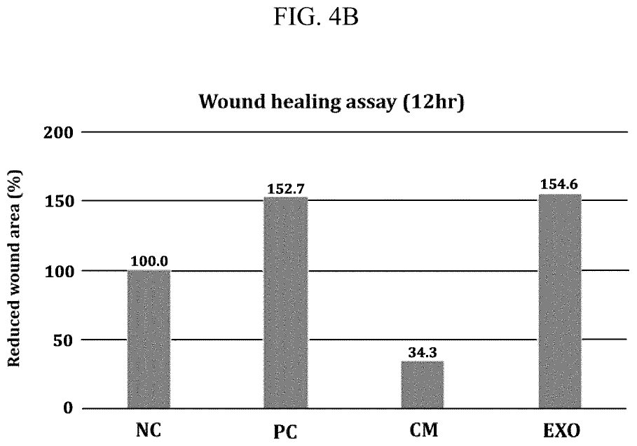

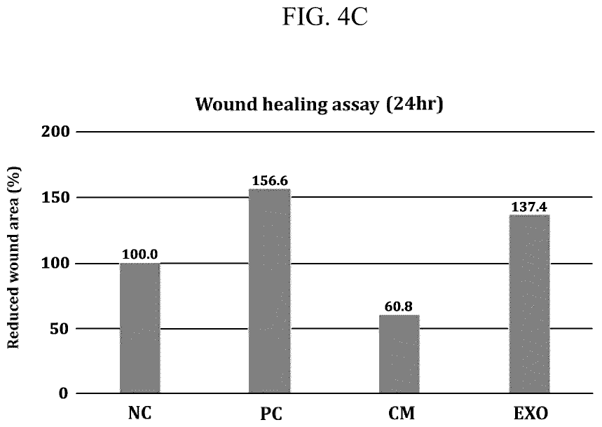

As a result of measuring the wound healing efficacy, it was confirmed that the treatment with the edelweiss-derived exosomes of the present invention remarkably increased the migration of the human dermal fibroblast compared to the positive control at 6 hours after the treatment, and also remarkably increased the migration of the human dermal fibroblasts compared to the negative control and the conditioned media of edelweiss callus at all the time points of measurement (at 6 hours, 12 hours and 24 hours after the treatment) (FIGS. 4A to 4C).

As can be seen from the above results, the edelweiss-derived exosomes of the present invention have an excellent effect for promoting the migration of human dermal fibroblasts, that is, an excellent wound healing or skin regeneration effect, as compared with the conditioned media of edelweiss callus.

Therefore, the edelweiss-derived exosomes of the present invention are useful as an active ingredient of a cosmetic composition for skin elasticity improvement, skin wrinkle reduction and/or skin regeneration and an active ingredient of a pharmaceutical composition for wound healing or wound healing acceleration.

### Example 6. Evaluation 1 of Anti-Inflammatory Effect of Edelweiss-Derived Exosomes

To evaluate whether the edelweiss-derived exosomes prepared as described in Example 2 exhibit an anti-inflammatory effect, RAW 264.7 cells were suspended in 10% FBS-containing DMEM, and then seeded into each well of a multi-well plate to reach a confluency of 80 to 90%. On the next day, the RAW 264.7 cells were treated for 24 hours with the edelweiss-derived exosomes diluted in a LPS-containing fresh medium (DMEM containing 1% FBS and 200 nM LPS). Experimental groups for evaluation of the anti-inflammatory effect were classified as follows:

(1) Negative control: an experimental group in which RAW 264.7 cells were treated with LPS alone;

(2) Positive control (PC): an experimental group in which RAW 264.7 cells were treated with LPS plus 200 μM dexamethasone; and

(3) Edelweiss-derived exosomes (EXO): an experimental group in which RAW 264.7 cells were treated with the edelweiss-derived exosomes, which were prepared in Example 2 and diluted with the LPS medium (treatment concentration of EXO based on the number of particles: 1.0×10⁹ particles/mL).

After the completion of culturing the RAW 264.7 cells of each experimental group, the culture supernatant was collected, and the inflammatory response was checked by measuring the inflammatory cytokines present in the culture supernatant. The amount of inflammatory cytokines in the culture supernatant was measured using an IL-6 ELISA kit. The amount of IL-6 (inflammatory cytokine) produced in the group treated with LPS alone, and the amount of IL-6 (inflammatory cytokine) produced in the respective group treated with LPS plus each of dexamethasone and the edelweiss-derived exosomes were measured using the ELISA kit (purchased from R&D system) according to the manufacturer's manual.

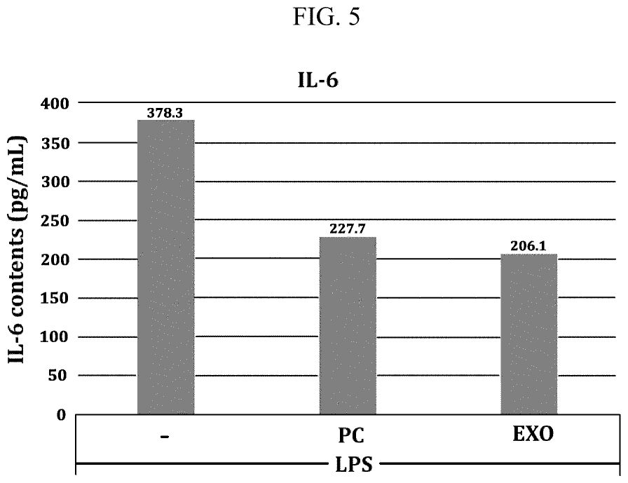

As shown in FIG. 5, it was confirmed that, when RAW 264.7 cells, which are mouse macrophages, were treated with LPS plus the edelweiss-derived exosomes of the present invention, the production of IL-6 was remarkably inhibited compared to that in the negative control group in which the RAW 264.7 cells were treated with LPS alone, and the production of IL-6 decreased to a level superior to that in the positive control group in which RAW 264.7 cells were treated with LPS plus dexamethasone.

As can be seen from the above results, the edelweiss-derived exosomes of the present invention have an excellent anti-inflammatory effect. Therefore, the edelweiss-derived exosomes of the present invention are useful as an active ingredient of a pharmaceutical composition for anti-inflammation, wound healing or wound healing acceleration.

### Example 7. Evaluation 2 of Anti-Inflammatory Effect of Edelweiss-Derived Exosomes

To evaluate whether the edelweiss-derived exosomes prepared as described in Example 2 exhibit an anti-inflammatory effect, RAW 264.7 cells were suspended in 10% FBS-containing DMEM, and then seeded into each well of a multiwell plate to reach a confluency of 80 to 90%. On the next day, the RAW 264.7 cells were treated for 24 hours with the edelweiss-derived exosomes diluted in a LPS-containing fresh medium (DMEM containing 1% FBS and 200 nM LPS). Experimental groups for evaluation of the anti-inflammatory effect were classified as follows:

(1) Negative control (NC): an experimental group in which RAW 264.7 cells were treated with LPS alone;

(2) Positive control (PC): an experimental group in which RAW 264.7 cells were treated with LPS plus 200 μM dexamethasone;

(3) Conditioned media of edelweiss callus (CM): an experimental group in which RAW 264.7 cells were treated with the conditioned media of edelweiss callus, which were prepared in Example 2 and diluted with the LPS medium (treatment concentration of CM based on the protein content: 50 μg/mL, 100 μg/mL and 500 μg/mL); and

(4) Edelweiss-derived exosomes (EXO): an experimental group in which RAW 264.7 cells were treated with the edelweiss-derived exosomes, which were prepared in Example 2 and diluted with the LPS medium (treatment concentration of EXO based on the protein content: 50 μg/mL, 100 μg/mL and 500 μg/mL).

After the completion of culturing the RAW 264.7 cells of each experimental group, the culture supernatant was collected, and the inflammatory response was checked by measuring the inflammatory cytokines present in the culture supernatant. The amount of inflammatory cytokines in the culture supernatant was measured using an IL-6 ELISA kit (purchased from R&D system). The amount of IL-6 (inflammatory cytokine) produced in the group treated with LPS alone, and the amount of IL-6 (inflammatory cytokine) produced in the respective group treated with LPS plus each of dexamethasone, the conditioned media of edelweiss callus and the edelweiss-derived exosomes were measured using the ELISA kit according to the manufacturer's manual.

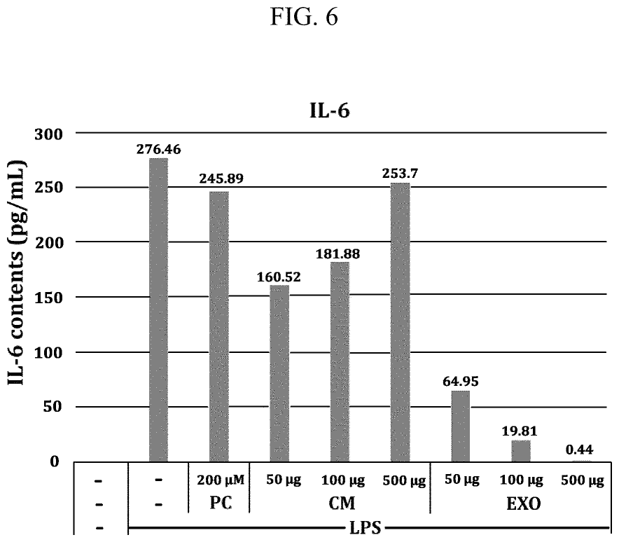

As shown in FIG. 6, it was confirmed that, when RAW 264.7 cells, which are mouse macrophages, were treated with LPS plus the edelweiss-derived exosomes of the present invention, the production of IL-6 was remarkably inhibited compared to that in the negative control group in which RAW 264.7 cells were treated with LPS alone, and the production of IL-6 also decreased to a level superior to that in the positive control group in which RAW 264.7 cells were treated with LPS plus dexamethasone.

In the group in which RAW 264.7 cells were treated with LPS plus the conditioned media of edelweiss callus, the production of IL-6 tended to increase as the treatment concentration of the conditioned media of edelweiss callus increased. In contrast, in the group in which RAW 264.7 cells were treated with LPS plus the edelweiss-derived exosomes, the production of IL-6 was remarkably inhibited compared to the group in which RAW 264.7 cells were treated with LPS plus the conditioned media of edelweiss callus. Especially, it was confirmed that the production of IL-6 tended to be inhibited in a concentration-dependent manner as the treatment concentration of the edelweiss-derived exosomes increased, thereby the edelweiss-derived exosomes have an excellent anti-inflammatory effect.

As can be seen from the above results, the edelweiss-derived exosomes of the present invention have an excellent anti-inflammatory effect. Therefore, the edelweiss-derived exosomes of the present invention are useful as an active ingredient of a pharmaceutical composition for anti-inflammation, wound healing or wound healing acceleration.

Although the present invention has been described with reference to the embodiments, the scope of the present invention is not limited to these embodiments. Any person skilled in the art will appreciate that various modifications and changes are possible without departing from the spirit and scope of the present invention and these modifications and changes also fall within the scope of the present invention.

We claim:

1. A method for preventing, suppressing, alleviating, ameliorating or treating inflammation of skin of a subject in need thereof, or healing wound or accelerating wound healing in the subject, the method comprising topically administering a composition to the skin, an inflammatory area or a wounded area of the subject, wherein the composition comprises exosomes derived from edelweiss as an active ingredient.

2. The method of claim 1, wherein the composition is topically administered to a target area of the skin, by injection, microneedling, spread, spray, transdermal delivery using a patch or a sheet, iontophoresis, or a combination thereof.

3. The method of claim 1, wherein the composition is an injectable formulation, an infusion formulation, a spray formulation, a liquid formulation, or a patch formulation.

4. The method of claim 1, wherein the exosomes are isolated from a conditioned medium of edelweiss plant cells, a conditioned medium of edelweiss callus, or a biological solution of edelweiss equivalent thereto, or derived from plant cells or plant stem cells of edelweiss.

5. The method of claim 1, wherein the exosomes are isolated and purified from a conditioned medium of plant cells obtained from a leaf or a stem tissue of edelweiss, or a conditioned medium of edelweiss callus induced by making a wound on a cotyledon germinated from an edelweiss seed.

6. The method of claim 1, wherein the subject is at least one selected from the group consisting of humans, dogs, cats, rodents, horses, cattle, monkeys and pigs.

7. A method for preventing, suppressing, alleviating, ameliorating or treating inflammation of skin of of a subject in need thereof, or healing wound or accelerating wound healing in the subject, the method comprising administering an effective amount of a composition to the skin, an inflammatory area or a wounded area of the subject, wherein the composition comprises exosomes derived from edelweiss as an active ingredient.

8. The method of claim 7, wherein the composition is administered to the subject by injection, microneedling, or a combination thereof.

9. The method of claim 7, wherein the composition is an injectable formulation, an infusion formulation, or a liquid formulation.

10. The method of claim 7, wherein the exosomes are isolated from a conditioned medium of edelweiss plant cells, a conditioned medium of edelweiss callus, or a biological solution of edelweiss equivalent thereto, or derived from plant cells or plant stem cells of edelweiss.

11. The method of claim 7, wherein the exosomes are isolated and purified from a conditioned medium of plant cells obtained from a leaf or a stem tissue of edelweiss, or a conditioned medium of edelweiss callus induced by making a wound on a cotyledon germinated from an edelweiss seed.

12. The method of claim 7, wherein the subject is at least one selected from the group consisting of humans, dogs, cats, rodents, horses, cattle, monkeys and pigs.

13. A method for skin elasticity improvement, skin wrinkle reduction or skin regeneration, the method comprising:
(a) (a1) applying a composition comprising exosomes derived from edelweiss as an active ingredient to a skin of a subject in need thereof; or (a2) contacting or attaching a patch, a mask pack or a mask sheet, which has the composition applied thereto or soaked therein, to the skin; or (a3) sequentially performing (a1) and (a2).

14. The method of claim 13, wherein the composition is a shampoo, a soap, a rinse, a surfactant-containing cleanser, a cream, a lotion, an ointment, a tonic, a treatment, a conditioner, a suspension, an emulsion, a paste, a gel, an oil, a wax, a spray, an aerosol, a mist or a power in step (a).

15. The method of claim 13, further comprising step (b) removing the patch, the mask pack or the mask sheet from the skin after step (a2) or (a3), and again applying the composition to the skin.

16. The method of claim 15, wherein the composition is a shampoo, a soap, a rinse, a surfactant-containing cleanser, a cream, a lotion, an ointment, a tonic, a treatment, a conditioner, a suspension, an emulsion, a paste, a gel, an oil, a wax, a spray, an aerosol, a mist or a power in step (b).

17. The method of claim 13, wherein the exosomes are isolated from a conditioned medium of edelweiss plant cells, a conditioned medium of edelweiss callus, or a biological solution of edelweiss equivalent thereto, or derived from plant cells or plant stem cells of edelweiss.

18. The method of claim 13, wherein the exosomes are isolated and purified from a conditioned medium of plant cells obtained from a leaf or a stem tissue of edelweiss, or a conditioned medium of edelweiss callus induced by making a wound on a cotyledon germinated from an edelweiss seed.

19. The method of claim 13, wherein the subject is at least one selected from the group consisting of humans, dogs, cats, rodents, horses, cattle, monkeys and pigs.
